(19) United States
(12) Patent Application Publication
(10) Pub. No.: US 2006/0057133 A1
(43) Pub. Date: Mar. 16, 2006

(54) Topical pathogenic-tissue-destroying liquid

(76) Inventor: Ramon Suarez Mendoza, Morelia (MX)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 10/508,595

(22) PCT Filed: Mar. 22, 2002
(86) PCT No.: PCT/MX02/00024

§ 371 Date: Jul. 11, 2005

(51) Int. Cl. A61K 38/54; A61K 33/00; A61K 31/47

(52) U.S. Cl. 424/94.2; 514/310; 424/718

(57) ABSTRACT

The invention relates to a method of producing a pharmaceutical composition, consisting in mixing nitric acid in composition with mucin and ptyalin enzymes which are used as absorbers. Together, said components produce a topical aqueous solution which destroys pathogenic tissue, by coagulating same, in order to treat a patient with warts, verrucas, skin cancer, tongue cancer, throat cancer, ulcerated tonsils, gingivitis, streptococcus or staphylococcus in the throat, uterine cervix cancer or fungus. The inventive composition can also be applied to any part of the skin or mucous membranes. Said composition is administered topically (externally) in one, two, three or four applications depending on the severity of the infection or wound. More specifically, the invention relates to a pharmaceutical composition comprising an effective quantity of nitric acid, mucin and ptyalin and distilled water which is used as an acceptable pharmaceutical vehicle or carrier. The composition is selective given that it strangulates the pathogenic tissue until said tissue is destroyed, leaving the healthy tissue intact apart from a slight mark which is caused by the effects of the nitric acid.

The present invention relates to the field of topical medications and is a topical liquid which destroys pathogenic tissue on the skin and the human mucous membranes, which is applied to healthy and diseased tissue, but only affects diseased tissue by coagulating same and thus completely eliminating it. The liquid is for external use and may be classified as an antiviral, antimicrobial, disinfectant and antimycotic.

### BACKGROUND OF THE INVENTION

There have been attempts to discover which genes are expressed in healthy tissue and which in diseased tissue, since the malfunctioning of the genes causes various diseases and if they could be identified, it would be possible to develop new strategies and pharmacological compositions which are effective against them.

William A. Haseltine stated this in his article entitled “Search for genes for the design of new medicines”, published in the Encarta encyclopaedia. The majority of human beings are familiar with the idea that a gene is something which transmits hereditary characteristics from one generation to the next. Perhaps what they did not know is that the cause of the majority of diseases, and not just hereditary diseases, is a malfunction of the genes. In cancer, arthrosclerosis, osteoporosis, arthritis and Alzheimer's disease, for example, specific changes occur in the activities of certain genes. Infectious diseases also usually cause the activation of some genes of the patient's immunological system. Ultimately, the accumulation of damage in the genes, as the result of a lifetime of exposure to ionising radiation and to harmful chemical agents, is probably related to changes which occur on aging.

Proteins direct all cell functions. They act as structural components, as catalysts in performing the multiple chemical processes of life and as control elements to regulate cell production and specialisation, and physiological activity at all levels. The development of a human being from a fertilised egg to the mature adult is ultimately the result of a series of ordered changes in the pattern of genetic expression in the various tissues.

Knowledge of which genes are expressed in healthy and diseased tissue would enable us, on the one hand, to recognise the deteriorations which occur during illnesses. We could therefore develop new strategies for the diagnosis of a number of diseases and create pharmacological compositions capable of modifying the activity of affected proteins or genes. Some of the identified proteins and genes could also be used by other investigators. A sort of molecular anatomy seems to have been considered.

It is proposed to identify the genes which are expressed in given tissue and to identify those which are of clinical interest. By this strategy, it is possible to generate a list of genes which are expressed in the affected tissue and to know how each of them is expressed, comparing them with those of a healthy tissue sample so the difference will reveal the genes (proteins) involved in the disease. Investigators can then produce the given human proteins for these genes in vitro.

Once the protein has been synthesised in its pure form, a test is prepared to detect the presence thereof in a patient, and pharmacologists can use the purified proteins to produce new pharmacological preparations. A chemical composition which inhibits the production of a protein present in a plaque may be considered as a pharmacological preparation.

It is stated that the disorders include autoimmune diseases which present when there is an impairment of the recognition mechanisms of the immunological system which reacts against the organism itself. Examples of them include: rheumatoid arthritis, myasthenia gravis, Hashimoto's disease, insulin-dependent diabetes mellitus, etc.

Infectious diseases which are due to viruses or bacteria and diffuse rapidly to numerous individuals include: exanthematic diseases (smallpox, scarlet fever, measles, rubella), influenza, cold, infectious mononucleosis, plague, haemorrhagic fever and sexually transmitted diseases (STD, Aids).

Many infectious diseases have been combated in the twentieth century on account of vaccines, antibiotics and the improvement in living conditions. Cancer has become a common disease, but many forms of the disease can now be effectively combated owing to the development of new techniques and numerous treatments. However, there is none which has a global nature for treating various types of cancer, whether internal or external.

Infection and infestation are a pathogenic contamination of the organism by external bacteriological agents (fungi, bacteria, protozoa, rickettsias or viruses) and by their toxins. An infection may be local—confined to a structure—or generalised and extending throughout the organism. The infectious agent penetrates the organism and begins to proliferate, and this triggers the host's immune response to this attack. This interaction generates the characteristic symptoms: pain, tumour (swelling), local flush (redness), functional impairments, increase in body temperature, tachycardia and leukocytosis.

The infectious agents penetrate the organism through various tracts. The most common are the respiratory, urinary and gastrointestinal tracts, but there are others: skin (especially if it is wounded), mucous membrane surfaces, ocular conjunctiva. Pregnant women can transmit diseases to their foetuses via the placenta. The probability and level of infection are related to the dose and virulence of the pathogenic agent and to the host's specific resistance and immunity to this microorganism. Resistance to infections is reduced in many diseases of the immunological system, in leukaemia and in cancer, and in cases of burns, severe lesions, malnutrition, senility, stress, drug addiction, corticotherapy, antineoplastic chemotherapy, etc. In healthy people, there are various groups of saprophytic bacteria, the natural bacterial flora, which form a line of defence against slight infections. Antibiotics, sulphamides and other pharmacological preparations may be used against serious infections. Vaccines are useful in preventing a considerable number of infectious diseases.

Penicillin, discovered by Alexander Fleming in 1928, is used in the fight against bacterial diseases. Penicillin is an important antibiotic derived from a mould and is effective against a wide range of bacterial diseases by destroying the bacteria and inhibiting its growth.

Many infectious diseases have been combated during the twentieth century by means of sanitation, antibiotics and vaccines, but actual pharmacological treatment began with the discovery by the German doctor, Paul Ehrlich, of arsphenamine, an arsenic compound used to treat syphilis. This was followed in 1935 by the announcement by the German scientist, Gerhard Domagk, that a dye, Prontosil red, was effective against streptococcal infections. The discovery of the active principle of Mercurochrome, sulphanylamide, led to the proliferation of the first group of significant pharmacological preparations: sulphamide bacteriostatic antibiotics. Penicillin was purified 10 years after its discovery and could therefore be widely used in medicine.

A treatment for tuberculosis was similarly discovered: streptomycin. When the bacteria became resistant, the combination of rifampicin with isoniazid emerged; this continued to be the preferred treatment. Hansen's disease (leprosy) is effectively treated by pharmacological preparations known as sulphones and malaria by quinine derivatives. No antibiotics have been found for diseases caused by viruses, but vaccines are becoming a key factor for prevention. Early vaccines included the smallpox vaccine discovered by Edward Jenner in 1796, the typhoid fever vaccine, the diphtheria vaccine discovered in 1923 and the tetanus vaccine discovered in the 1930s.

In the 1930s, American microbiologists developed a method for growing viruses in tissue cultures, which represented a great advance in the preparation of vaccines against viruses. This discovery led to vaccines against yellow fever, poliomyelitis, measles and rubella. In the early 1980s, genetic engineering led to the development of vaccines against hepatitis B, hepatitis C, influenza, herpes simplex and chicken pox, and a vaccine against malaria was tested.

The fight against infectious diseases was complicated in the latter part of the twentieth century by the increase in the resistance of the microorganisms to antibiotics and the discovery of new diseases such as legionnaire's disease and acquired immunodeficiency syndrome (AIDS).

The resistance of bacteria to antibiotics, owing to genetic mutation, is a growing problem nowadays. The excessive and inappropriate use of antibiotics has caused microorganisms to become resistant thereto and has led to the appearance of new, more virulent strains of some pathogenic microorganisms, which have spread rapidly. Some bacteria which cause serious infections in hospitals have become resistant to antibiotics which were used as the last resort.

These include golden staphylococcus, which is resistant to the antibiotic, methicillin, since similar antibiotics have been used in recent years to promote the increase in weight of farm animals reared on a large scale. At present, there is no treatment for infections caused by these bacteria. The bacteria are unicellular organisms which do not have a differentiated nucleus. Although the majority are harmless, approximately 200 are pathogenic, in other words able to cause serious diseases such as cholera, tuberculosis, leprosy and pneumonia, mainly by producing toxins or destroying tissues.

Cancer

The mortality due to various types of cancer has increased in recent years. Some aspects of this disease have not yet been explained scientifically, even though it is known that occupational and environmental exposure to chemical products are some of its causes. In particular, the consumption of tobacco causes the majority of lung cancers and some bladder, mouth, throat and pancreas cancers. Early diagnosis, in particular of cervical cancer, helps to reduce mortality. The first treatment applied was radiation, but pharmacological treatment was introduced in the 1960s. Pharmacological treatment currently cures many cases of breast and testicular cancer and some cancers which affect the blood, especially in children. Investigators began to study the efficacy of certain substances known as cytokines (interferon) with anti-cancer pharmacological preparations.

Herpes

Herpes (to creep), the generic name of various types of cutaneous eruption caused by the most significant human pathogenic viruses. Their main representatives are: the herpes simplex virus type 1 and type 2 and the varicella-zoster. Other significant herpes viruses are the Epstein-Barr virus, which causes infectious mononucleosis, and the cytomegalovirus, which can produce congenital abnormalities when it affects women during gestation.

Herpes Simplex Virus

The herpes virus type 1 causes feverish blisters associated with various feverish infectious diseases (colds, influenza, pneumonia). The blisters appear around the lips and in the mouth (also known as labial herpes); on the nose, face and ears, and in the buccal and pharyngeal mucus. It has been possible to isolate the virus from the neuronal bodies of the facial nerve during the period between eruptions: this is its reservoir. There is no curative treatment; topical pharmacological compositions may be applied to alleviate the pain the irritation and/or the inflammation.

The herpes simplex virus type 2 causes genital herpes. This is an increasingly widespread, sexually transmitted disease. Sometimes it is accompanied by severe headaches and fever. It begins with moderate local pruritus, followed by the progressive eruption of vesicules. These burst, form scabs and eventually dry. This entire process can last from 1 to 3 weeks. New vesicular eruptions often appear while the previous eruption is drying. Another transmission pathway is the neonatal route: the neonate of a diseased mother is infected as it passes through the birth canal, contracting the systemic disease, which is usually fatal. These children have to be born by Caesarean section on account of this serious risk. Genital herpes has been treated topically since 1982 and by a systemic treatment since 1984. The herpes virus type 2 causes cancer of the cervix (neck of the uterus): the viruses lodge in the cells of the mucous membrane and eventually, years later, sometimes cause cancerous transformation of these cells. The viruses can also affect the central nervous system, particularly in patients who are debilitated or have compromised immunity, such as those suffering from cancer, and this causes serious encephalitis. Early treatment can prevent death or serious cerebral consequences.

Verruca

Verruca, a small circumscribed benign tumour of the outermost layer of the skin. Verrucas are flat or raised above the surface of the skin surrounding them and have a firm consistency. They are caused by the human papovavirus, have different sizes and are sometimes painful, particularly if they are located on the feet (plantar verrucas).

The treatment involves the use of topical pharmacological compositions. If the verruca is spreading, it may be treated by freezing with dry ice, X-rays, thermocautery with an electric scalpel or surgical resection.

Gingivitus

Painless inflammation or degeneration of the gum tissues. It may begin in puberty, but it usually appears in adults, usually as the result of deficient buccal hygiene. People who suffer from certain diseases, such as diabetes mellitus or acquired immunodeficiency syndrome (AIDS) are more likely to develop this disorder.

It is caused by the increase in the bacterial plaque, a film of bacteria which attaches itself to the teeth at the gum line. The toxins released by these microorganisms irritate the gums and cause them to become inflamed and bleed, and this enables bacteria to penetrate inside the gum line. If it is not treated, the inflammation not only damages the gum, but also destroys the bones supporting the teeth. Finally, the gums separate from the teeth, which may begin to fall out.

The treatment involves thorough professional cleaning of the teeth, to eliminate bacterial plaque, in other words the use of surgery.

Nitric Acid

Corrosive colourless liquid whose chemical formula is HNO₃. Medieval alchemists knew it as aqua fortis (strong water). Nitric acid is obtained commercially by the action of sulphuric acid on sodium nitrate. It may also be prepared by catalytic oxidation of ammonia. It is a strong acid and a powerful oxidising agent. It produces yellowish colouring on the skin when it reacts with certain proteins and forms yellow xanthoproteic acid. Commercial concentrated nitric acid contains approximately 71% of HNO₃, the remainder being water. Fuming nitric acid, which is also used commercially, is composed of nitric acid and gaseous nitrogen oxide in solution. It has a reddish or dun colour and is more active than other forms of nitric acid. Common and fuming nitric acid have numerous applications. They are used in chemical synthesis, in the nitration of organic material to form nitrogenous compounds and in the production of inks and explosives. Nitric acid has a melting point of −42° C. and a boiling point of 83° C. Almost all nitrates are soluble in water. One exception is bismuth nitrate which is used in medicine for treating intestinal disorders.

Staphylococcal Infections

Gram-positive spherical staphylococcal cells are usually found in irregular groups of resembling bunches of grapes. Some of them are members of the normal flora of the skin and mucous membranes of human sera and others cause suppuration, the formation of abscesses, a variety of pyogenic infections and even fatal septicaemia. Pathogenic staphylococcus sometimes haemolyses blood, coagulates the plasma and produces a variety of extracellular enzymes and toxins. The most common case of food poisoning is caused by a heat-stable staphylococcal toxin.

The Staphylococcus genus has at least 30 species. The three main species of clinical significance are Staphylococcus aureus, epidermidis and saprophyticus. Staphylococcus aureus is coagulase-positive and differs from the other species. Staphylococcus aureus is the major pathogen for humans. Almost everybody has some type of infection by staphylococcus aureus in their life, varying in severity from food poisoning or severe skin infections to severe infections which endanger the life. Coagulase-negative staphylococci are normal in the human flora and sometimes cause infection with implants of devices, especially in very young patients. Approximately 75% of infections caused by coagulase-negative staphylococcus are due to Staphylococcus epidermidis. Infections due to staphylococcus Warneri, staphylococcus hominis and other species are less common. Staphylococcus is a relatively common cause of urinary tract infections in young women. Staphylococcus produces catalase, and this is what distinguishes it from staphylococcus.

The colonisation by staphylococcus aureus of the articular cartilages, of which the main component is collagen, which appears in the space between joints, is an important factor in the development of septic arthritis. Remains of haematogenically acquired bacterial arthritis currently represent a serious medical problem. The arthritis progresses rapidly, destroys the joints and is difficult to eradicate. Typically, less than 50% of infected patients do not recover without serious damage to the joints. Staphylococcus aureus is the predominant pathogen isolated from adult patients with haematogens and secondary osteomyelitis.

In infections associated with staphylococcus, plastic medical implants with metal surfaces become covered with host plasma and protein matrix, such as fibrinogen and fibronectin, soon after implantation. The capacity of staphylococcus aureus and other staphylococcal bacteria to adhere to these proteins is an essential reason for the investigation.

Due to the significant increase in the isolated staphylococcus which is resistant to most antibiotics, organisms which are resistant to the new antibiotics are rapidly emerging. With the introduction of each new antibiotic, staphylococcus has been capable of counting on β-lactamase, altered penicillin-bound proteins, and membrane proteins of mutated cells which are capable of withstanding the bacteria. Consequently, staphylococcus aureus resists methicillin (MRSA), and multidrug-resistant organisms have emerged and established a strong hold in hospitals and clinics for the elderly throughout the world.

Nowadays, more than half of the staphylococcal species which cause hospital infections are resistant to all antibiotics, with the exception of vancomycin, and it seems to be merely a question of time before vancomycin will also be ineffective.

There is an urgent growing need for therapies to treat staphylococcal infections such as staphylococcus aureus, which are effective against antibiotic-resistant species of bacteria. The Health Institute in the United States of America has recently indicated that this is its aim and that it is now a national priority.

Radioimmunotherapy

Raidoimmunotherapy generally employs radionuclei, and is more specifically related to immunotherapy using radionuclides which emit α (helium nuclei), β and γ particles.

WO 90/15625, published on 27 Dec. 1990, relates to radioimmunotherapy using α-particles emission. More particularly, it relates to the use of actinium or one of its daughters in radioimmunotherapy. Also provided are immunoconjugates comprising an isotope, a chelating agent and a slowly localising antibody. The application includes pharmaceutical compositions comprising said immunoconjugates. The compositions according to the invention are useful for the treatment of micrometastases in adjuvant therapy, as well as for treatment of localised tumours.

The cytotoxicity of α-particles is attributed to the high linear energy transfer (100 keV/μm) and the high electrical charge of the particles.

Other radioimmunoconjugates which are radionuclides are used, such as 212 bismuth, coupled to a monoclonal antibody by the cyclic anhydride of diethylenetriamine pentoaacetic acid (DTPA). The monoclonal antibody is directed against a murine antigen, which is present on both normal and malignant murine T-cells. Another α-particle emitting isotope for use in immunotherapy is ²¹¹astatine. This leads to problems in the handling of the parent isotopes such as ²²⁸thallium, ²²⁴radium or ²¹²lead.

The same application mentions that the best solution to the problem would be the use of human monoclonal antibodies, but these are not currently available, and European patent application No. 0151030 produces IgM antibodies, the problem with these being that they are very slow in reaching the site in the body where their antigens are located (the tumour). It took them from one day to several days.

Mexican Patent 172,967, granted on 26 Jan. 1994 and entitled “Process for obtaining new mixed copper amino acid complexes based on phenanthroline and alkyl derivatives thereof as anticancer agents”. The inventor, Lena Ruiz Azuara, describes a process for obtaining new mixed copper amino acid complexes as anti-cancer agents in therapeutic use for treating solid or liquid cancerous tumours such as leukaemia; the complexes obtained are of the type which employ nitrogen in the form of phenanthroline-based nitrates and aromatic alkylated derivatives thereof with anti-cancer properties of the formula [CU (N—N) (N—O)]^{+ -}NO₃, wherein the bond N—N (diimine) corresponds to 1-10 phenanthroline and the alkyl derivatives thereof, the bond N—O corresponds mainly to one of the thyrosimalamine, threonine, tryptophan, valine, isoleucine, cysteine, diglycin, phenylalanine, glycine, histidine, serine, tyrosine, asparagine, alanine or phenylalanine, amino acids or ions. The process is characterised in that it comprises the steps of: reacting an aqueous solution based on an aliphatic alcohol and 4,7-diphenyl-1,10-phenanthroline with a copper compound, preferably CuNO₃.5H₂O at ambient temperature; the product obtained is then reacted in an aqueous solution of amino acid while adjusting to a slightly alkaline pH.

A type of drug, in which there is a correlation between biological activity and structure, is that of the chelates of metals, such as iron, ruthenium, cobalt, manganese, zinc and copper. Chelators can be designed to inactivate bacteria, viruses and fungi by capturing the metal ions required for the metabolism of these microorganisms. Metal ions which are toxic to them may also be administered.

The term ‘mixed complex’ means any coordination compound with two or three bonds of the chelating type with are different from one another and exclude the solvent of the category of the chelate bond.

It has been found in many cases that the enzyme action depends on an elemental process in which the enzyme-substrate complex is generated and has characteristics similar to those of a mixed complex. Specific metal chelates, administered in low concentrations, are active against some bacteria, fungi, viruses and some tumour cells. It is thus known that Staphylococcus pyogenes has become very resistant to the action of many standard antibiotics and succumbs in the presence of saturated chelate complexes.

The process developed in this patent for obtaining mixed copper amino acid complexes based on phenanthrolines and the alkyl derivatives thereof as anti-cancer agents enables highly water-soluble crystals to be obtained and the reaction takes place at ambient temperature, thus preventing the therapeutic properties of the amino acid obtained from deteriorating.

Mexican Patent 9910447, filed on 12 Nov. 1999, for an “Antiviral pharmaceutical composition comprising glyceric acid and at least one protein endowed with antiviral activity” resides in the field of antivirals. This composition mainly treats infections caused by the herpes simplex virus, type 1, which causes facial and oropharyngeal lesions. In the past, herpes simplex virus, type 1, infections were treated with vidarabine, but this has been almost completely replaced by acyclovir, owing to its toxicity. Acyclovir is consequently the most widely used pharmaceutical composition for the treatment of buccal and labial lesions (eruption due to fever). However, the topical use thereof often leads to burns and irritation or the mucous membranes. Furthermore, acyclovir is completely efficient when administered during the first infections, but is not very effective in the case of recurrent infections and therefore it does not resolve or prevent reinfection by herpes simplex virus type 1.

In addition, the treatment has the disadvantage of causing side effects such as nausea, diarrhoea, irritation, headaches, renal inadequacy and nephrotoxicity.

Consequently, there is a real need for an active medication for the treatment of herpes simplex virus type 1 infections, even in the case of recurrent infections, and which must also be free from side effects.

In reality, it is also known that glyceric acid demonstrates some antiviral activity, enormously inhibits the synthesis of viral glycoproteins and, only in very high doses, also inhibits the synthesis of cellular glycoproteins. In fact, although the action of glyceric acid in protein synthesis, in both normal and infected cells, is practically irrelevant even at doses of 4 mM, the synthesis of glycoproteins demonstrates a substantial difference in normal and infected cells. At 4 mM, the synthesis of glycoproteins in normal cells is also slightly affected, but production of the virus is inhibited by 99%. The experimental results show that there is a great reduction in the infection and that the cells retain their cellular integrity.

An object of the invention in question is to provide a pharmaceutical composition which is characterised in that it comprises glyceric acid and at least one protein that has antiviral activity, the protein being selected from the group comprising lysozymes and lactoferrins. Normally, the pharmaceutical composition of this invention may be used in the treatment of topical viral infections. Preferably, the virus is of a herpetic type, in particular the herpes simplex virus type 1 (HSV1). The pharmaceutical compositions of this invention are prepared in the form of creams, ointments and medicated plasters for topical administration.

The optimum dosage of this invention will preferably be such that it allows daily administration of 0.25-8 mg/ml of glyceric acid, 0.5-10 mg/ml of lysozyme and/or 0.1-4 mg/ml of lactoferrin.

Mexican Patent application No. 960585, filed on 18 Dec. 1996, relates to the isolation and characterisation of amino acid and nucleotide sequences of a new member of the genetic family of mucins. The invention relates, in particular, to the provision of reagents for the diagnosis of patients and for vaccination in the treatment of specific diseases by stimulation of the immune defence.

The epithelia of the respiratory, reproductive and gastrointestinal tracts of superior organisms are covered by a protective secretion known as mucus. This mucus gel is composed of up to 95% of water and up to approximately 5% of mucins. The mucins are glycoproteins with two specific characteristics: firstly, at least 50% of their molecular weight consists of oligosaccharides which are bound by the C-glycoside to theronine radicals and serine from the protein skeleton and, secondly, this strongly glycosilylated region is composed of repetitive sequential units. Mucins may be divided into two groups, on the one hand, the secretory mucins which, by means of intermolecular bisulphide bridges, appear in form of oligomers and, on the other hand, the mucins fixed in membranes which are attached to the plasma membrane via a hydrofuge region.

Changes in the post-translational expression and modification of the mucins are associated with various diseases, such as cancers, cystic fibrosis, ulcerative colitis and Crohn's disease, and may make the mucins the subject of clinical investigations. The antibodies which recognise mucin epitopes are used as markers in diagnosis.

Consequently, the aim of the invention in question is to find other mucins, in combination, the value of the mucins in diagnosis and thus to make an essential contribution to the diagnosis of tumours.

It is mentioned in application 9606585 that the DNA fragments which encode the mucin MUC8 may be inserted in eukaryotic expression vectors and may be used for the (stable or transitory) transformation of mammalian cells, especially human cells. This idea is based on reintegrating in patients the transfectants which express large amounts of MUC8 to bring about a specific immune response associated with tumours by recognition of antibodies or CTL. A further aspect is the detection of tumours or inflammations in patients by the detection of mucin antigens in tissues or sera.

The MUC8 polypeptides which can be isolated after prokaryotic or eukaryotic expression can be applied directly as protective factors in various diseases (ulcers) or can be used to generate further specific MUC8 antibodies. If the polypeptides are greater than 10 KD, they can be used directly as immunogens, otherwise it is necessary to couple them to carrier proteins for effective immunisation.

For use in cancer therapy, the specific MUC8 antibodies, which are as humanised as possible, may be coupled to toxins or radionuclides and administered to patients whose tumoral tissue exhibits increased MUC8 expression, to specifically harm or mark the tumoral tissue.

Mexican Patent application No. 9603535, filed on 21 Aug. 1996, discloses the use of nitric acid as an antimicrobial agent and discloses a form of dose for the use thereof in the treatment of bacterial, viral and fungal conditions, in which the form of dose may be a pharmaceutically acceptable vehicle and comprise an acidifying agent adapted to reduce the pH of the ambient medium.

The application refers to acid nitrite as an antimicrobial agent. Although the nitrite has been used as a food preservative for many years, it has been found that the nitrite, in a low concentration, is effective in reducing bacterial, fungal and viral populations in the animal body. It is believed that this mechanism is used by mammals to destroy ingested microorganisms.

Active entero-salival circulation in man causes a continuous flow of nitrate toward the mouth, where it is rapidly reduced to nitrite by the bacteria located on the tongue.

It has been found that the exposure of a yeast and the E. coli bacterium to concentrations of nitrite in the saliva, together with acidic conditions similar to those found in the stomach, for 1 hour, causes a reduction in the survival thereof, depending on the dose. Therefore, it is obvious that, with the generation of nitrogen oxides and/or nitrous acid in the mouth and in the gastrointestinal tract, in particular the upper gastrointestinal tract, the acidic nitrite prevents microbial infection. The salvia provides a continuous stream of nitrate to the upper gastrointestinal tract. The oral conversion of nitrate to nitrite is rapid and is restricted to the surface of the tongue in man.

The above-identified mechanism may also be applied to the destruction of microorganisms on the skin, for example athlete's foot or tinea pedis. The nitrogen oxides are effective in destroying infectious organisms on the skin, including fungi, yeasts, bacteria and viruses. They cause moderate erythema (redness) of the skin owing to the release of nitric oxides, but they do not cause significant inflammation. Nitric oxide readily diffuses through all the cell membranes and has high affinity for respiratory enzymes which contain iron-sulphur, and damages the bacterial DNA. When it is produced enzymatically by activated leukocytes, nitric acid destroys Leishmania sp, staphylococcus sp, Francisella sp, etc.

Mexican Patent application No. 9700312, filed on 10 Jan. 1997, describes various compositions and methods for use in achieving specific coagulation of the blood. This is embodied by the specific coagulation in vivo of the tumour vasculature causing regression of the tumour through the application to a site of a coagulant, which is a bi-specific antibody in this case. In other words, the application refers generally to the fields of blood vessels and coagulation. More particularly, it provides a variety of immunological reagents based on the growth factor, including bi-specific antibodies, for use in achieving specific coagulation.

Significant progress has been made with new chemotherapy agents for the treatment of some tumours, more particularly the development of regimes for the concurrent administration of pharmaceutical compositions, and this represents a significant advance at cell and tissue level. Despite the advances which have been made in some tumours, many of the most prevalent forms of human cancer still resist effective chemotherapy intervention. A significant problem to be resolved in any treatment regime is the concept of “total cell destruction”. This concept maintains that an effective treatment regime necessarily leads to total cell destruction of all so-called malignant cells, in other words the cells which have the capacity to increase uncontrollably and to replace any tumoral mass which may have been removed. Owing to the ultimate need to develop therapeutic agents and regimes which lead to total acceleration, certain types of tumour have been more susceptible than others to therapy. For example, soft tissue tumours (lymphomas) and tumours of the blood and other blood-forming organs (leukaemia) have generally responded better to chemotherapy than solid tumours such as carcinomas.

The increase in the dose of chemotherapy agents frequently leads to toxic side effects, and this creates limits to the efficacy of conventional antitumour agents.

The strategy for developing successful antitumour agents involves the design of agents which selectively destroy tumour cells while having relatively few adverse effects, if any, against normal or healthy tissue. This objective has been difficult to achieve because there are few qualitative differences between neoplastic and normal tissues. Therefore, investigations have concentrated on the identification of “marker antigens” which are specific to the tumour and can act as immunological targets. Unfortunately, specific tumour antibodies do not themselves have sufficient antitumoral effects to make them useful in cancer therapy.

Immunotoxins with selective direction, typically an antibody or fragment directed to the tumour, with a cytotoxic agent have been used more recently. It has been found that the immunotoxins are effective for the treatment of lymphomas and leukaemias. However, lymphoid neoplasias are particularly susceptible to immunotoxin therapy because tumour cells are relatively accessible to immunotoxins which emerge from the blood.

In contrast to their efficacy with lymphomas, it has been found that immunotoxins are relatively ineffective in the treatment of solid tumours. The main reason for this is that solid tumours are generally impermeable to molecules having the size of the antibodies. A further significant problem is that antigen-deficient mutants can escape and be destroyed by the immunotoxin and can grow again. The antibodies entering the tumour mass are not uniformly distributed owing to the dense packing of the tumour cells and of the fibrous stromas, which both form a formidable physical barrier to macromolecular transport.

Consequently, it is very obvious that there is a significant need for the development of novel strategies for the treatment of solid tumours. One approach involves the directing of agents or medications which affect the tumour vasculature rather than the tumour cells. The growth of the solid tumour depends greatly on the vascularisation of the tumour, and the growth of the tumour cells can only be maintained if the supply of oxygen, nutrients and other growth factors and the flow of metabolic products are satisfactory.

Medications or antibodies are required, which recognise the endothelial cells of the tumour but do not attack those of healthy or normal tissues.

It seems that it is better to direct the solution of this problem toward the tumour vasculature, the solution having a high degree of specificity, no side effects and causing definitive damage to the tumour vasculature and therefore causing an avalanche of death to the tumour cells. As an additional advantage, is should be inexpensive and easy to handle clinically. The following detailed description of the present invention will pursue these advantages.

Application No. 9700312 provides novel compositions and methods for use in achieving specific coagulation in the tumour vasculature, with limiting side effects. It is achieved by the use of bi-specific immunological compositions based on the growth factor, which are capable of stimulating coagulation in the vasculature associated with the disease, and by methods for the preparation and use thereof. The invention provides fixing bonds which can generally be described as “bi-specific fixing bonds” which fix to a target cell related to the disease, such as a tumour cell, or a component associated with this cell.

### BRIEF DESCRIPTION OF THE INVENTION

The object of the present invention is to protect a selective pharmaceutical composition which comprises a quantity of nitric acid, mucin and ptyalin, and a pharmaceutically acceptable vehicle.

A further object of the present invention is to protect a selective medication for the treatment of streptococcal and staphylococcal throat infections.

An additional object of the present invention is to protect a selective medication for the treatment of viral infections of the human skin, such at verrucas and warts.

It is also an object of the present invention to provide a selective topical antiviral liquid which prevents the growth of the virus that produces the affliction.

Yet a further object of the present invention is to provide a selective anti-microbial topical liquid which eliminates the microbes which cause the infliction.

It is also an object of the present invention to provide a pharmaceutical composition, or selective topical liquid which does not cause side effects when used to treat the infection.

It is also a further object of the present invention to provide a selective pharmaceutical composition which regenerates the damage tissue after the infection.

A further object of the present invention is to provide a selective liquid pharmaceutical composition which enables pathological human or animal tissue to be removed without the need for thermocautery or surgery.

A further object of the present invention is to provide a pharmaceutical composition having a high degree of specificity which definitively amply eliminates tumour cells.

A further object of the present invention is also to provide a pharmaceutical composition of topical liquid which is easy to apply and convenient for clinical use.

Yet a further object of the present invention is to provide a pharmaceutical composition which recognises and eliminates tumour cells, but not healthy or normal tissue cells.

A further object of the present invention is to provide a pharmaceutical composition or topical liquid which affects the tumour vasculature by coagulating it.

A further object of the present invention is to provide a pharmaceutical composition which is effective in treating external solid tumours and tumours of the mucous membranes, which may be treated topically and fibrous tumour stromas.

Yet a further object of the present invention is to produce a medication which is used topically on the eyelids, cervix, penis, anus rim, throat, gums, tongue, skin, scalp, benign verrucas, sole of the feet, calluses and nail fungi.

### DETAILED DESCRIPTION OF THE INVENTION

The aqueous or liquid pharmaceutical solution described hereinafter may be considered as a poison for the tumour.

Poison

Poison, any substance which produces a disease in the living organism, or tissue lesion or which interrupts natural life processes when it comes into contact with the organism. The majority of poisons taken in sufficient quantities are fatal. A poisonous substance may be of mineral, vegetable or animal origin, or produced in the laboratory, and can assume the form of a solid, liquid or gas. Poisons may classified as corrosive, irritating or narcotic. The latter are known as systemic or nerve poisons.

Corrosive poisons include strong acids or alkalis which cause external or internal tissue destruction, in other words abrade the skin or the gastric mucous membranes or mucous membranes of other organs. Normal poisons, which are known as corrosive agents, include hydrochloric acid, carbolic acid, mercury bichloride and ammonia.

Irritants such as arsenic, mercury, iodine and laxatives, act on the mucous membrane, causing gastrointestinal irritation and inflammation accompanied by pain and vomiting. Diluted corrosive poisons also have these effects. The irritants include accumulative poisons, which are those substances that are absorbed gradually without causing an obvious lesion until they suddenly produce their effect.

Blood poisoning, which is also of a bacterial nature, is caused when a virulent microorganism invades the circulation of the blood through a wound or an infection. Symptoms include shivering, fever, prostration and frequently secondary infections or abscesses in various organs and the skin. The majority of gaseous poisons also affect the blood. As these gases restrict the organism's capacity to absorb oxygen, they are usually included in the category of asphyxiants, this group including the known carbon monoxide. However, there are also corrosive and irritating gaseous poisons.

The present invention describes the method of preparing a completely novel pharmaceutical composition for eliminating human and animal tumour tissues, abnormal or diseased tissues, without the need to use acids which are corrosive in the natural state and burn, abrade and therefore also destroy healthy tissue. With this aqueous composition, it is not necessary to employ thermocautery to eliminate pathogenic tissue, and surgery is not required either. This composition respects healthy tissue and permits the complete regeneration thereof by addition.

The pharmaceutical preparation coagulates diseased or abnormal tissue, affecting its vasculature or tissue nutrition pathways containing or accommodating it, such as warts, verrucas, cancerous tissue, some dark patches on the skin, inflamed tissue or tissue infected by fungi; throat infections caused by haemolytic β streptococcus, staphylococcus aureus, etc., thus eliminating the presence of rheumatic fever in patients diseases with this type of infection. It also eliminates granular tonsillitis, gingivitis and pharyngitis, in which granulation conceals the microbes causing the disease. It prevents the dissemination of fungi, microbes and the toxins thereof. It eliminates skin, tongue, throat and cervical cancer, generally where it is intended to apply this topical liquid composition according to the present invention.

Sometimes, when the cancer has begun to destroy the nose, the tongue and the lower eyelid, use of the topical liquid to eliminate cancerous tissue leads to regeneration of the tissues in the injured regions and does not leave a mark or a scar.

In the case of granular tonsillitis and pharyngitis, which are caused by haemolytic β streptococcus and staphylococcus aureus, which emerge from the infected zone and cause rheumatic fever and invade the joints where they lead to rheumatoid arthritis. Streptococcus and staphylococcus lead to isolated granulation of the circulation in the throat which is why they prevent the admission of any medication which might affect or eliminate them. These afflictions frequently damage the heart valves. The topical liquid forming the subject of the present invention coagulates the infected tissue by affecting its vasculature and eliminates it together with the granulations concealing the viruses or microbes. In addition to effecting a cure, this prevents the dissemination of microbes and toxins which cause rheumatic fevers and rheumatoid arthritis, so children afflicted with throat infections will be eliminated from the statistics.

The topical liquid or aqueous composition is composed of substances such as nitric acid and enzymes, mucins and ptyalin which are combined to form an aqueous pharmaceutical composition which is applied to the infected tissues and selectively attacks the cells of the diseased tissue or tumour. This composition is applied to the tissues which are to be eliminated, in dabs which are applied using a cotton swab moistened with the topical liquid that destroys the pathogenic tissue. It is applied to the diseased tissue as necessary, until the tissue changes colour and a small clear or pink halo appears round the treated tissue. The site is then washed with clean water. When applied to the mucous membranes, washing after application of the pathogenic-tissue destroying topical liquid is not necessary. Slight heat is felt when the composition is applied, but disappears shortly afterward or the next day. However, this sometimes depends on the sensitivity of the individual (only individuals who have a tendency to form keloid scars will be left with a slight sign).

When cancerous tissue is to be removed by traditional methods (surgery and thermocautery), eradication or burning of the tissue also stimulates it and generally causes metastasis. With the liquid according to the invention, however, the tissue coagulates immediately and it is virtually impossible for a malignant cell to escape and propagate in the organism and emerge in further tissue.

Nitric acid appears to be the least aggressive toward the skin and mucous membranes, and penetrates deeply into skin tissue, in contrast to sulphuric and hydrochloric acid which are too aggressive for the tissues. On the other hand, citric acid had very weak action and did not succeed in coagulating the diseased tissue.

Gastric and pancreatic enzymes have been used, but they were very aggressive, left scars and burnt the tissue, so it was necessary to reject this type of enzyme when preparing the mixture forming the pathogenic tissue-destroying topical liquid.

The mixture is prepared in the following manner:

In a preferred embodiment, mucin and ptyalin are used as starting materials and are diluted separately in a volume of water in a proportion of 50% of distilled water and 50% of enzymes. On the other hand, the nitric acid was diluted in a proportion of 5% to 20% of volume of distilled water. The foregoing process was carried out at ambient temperature and under acceptable septic conditions for preparing a mixture.

In other words, for a volume of 100 units, 5 to 20 units of distilled water are mixed with 40 to 90 units of nitric acid, plus 5 to 20 units of diluted mucin, plus 5 to 20 units of diluted ptyalin, to make up the required total of 100 units of volume.

In a 100 ml preparation, 5 to 20 ml of distilled water are mixed with 40 to 90 ml of nitric acid, plus 5 to 20 ml of diluted mucin, plus 5 to 20 ml of diluted ptyalin to make up the required total volume of 100 ml.

A preferred embodiment of the invention involves mixing a volume of 60 ml of nitric acid with 20 ml of water, plus 10 ml of diluted mucin, plus 10 ml of diluted ptyalin.

A further preferred embodiment of the invention involves mixing 80 ml of nitric acid with 5 ml of water, then with 5 ml of diluted mucin plus 10 ml of diluted ptyalin.

Yet a further preferred embodiment of the invention involves mixing 50 ml of nitric acid plus 20 ml of water with 15 ml of diluted mucin plus 15 ml of diluted ptyalin.

A further preferred embodiment of the invention involves mixing a volume of 70 ml of nitric acid with 10 ml of water, with 10 ml of diluted mucin plus 10 ml of diluted ptyalin.

Yet a further preferred embodiment of the invention involves mixing 40 ml of nitric acid with 20 ml of water, with 20 ml of diluted mucin and 20 ml of diluted ptyalin.

It should be emphasised that the use of mucin is to stimulate the formation of antibodies in a mammal. The two enzymes, mucin and ptyalin, are also used as an absorber to reduce the corrosive power of the nitric acid.

The aqueous pharmaceutical composition according to the present invention is fixed to the tumour vasculature cell surface and is capable of effecting coagulation in the vasculature associated with the disease. The fixing of the composition to a tumour vasculature component is induced by an enzyme, such as mucin and ptyalin, this induction being promoted by the power of penetration of the nitric acid into the dermis.

The liquid pharmaceutical composition is fixed to the cell body of the tumour vasculature dermis and coagulates the stroma components of the malignant tumour, is fixed to a basic membrane component or to a platelet, and also to a diseased cell or to a component of the environment associated with the tumour and to a tumour cell surface receptor.

It is fixed to the endothelial cell surface of the tumour vasculature and immediately opportunely binds with a coagulation factor dependent on nitric acid, mucin and ptyalin.

One of the above-described dilutions of the pharmaceutical composition is applied to the dermis of the eyelids, cervix, penis or anus rim; another of the dilutions is for application to the throat, gums or tongue; another dilution is for the skin, scalp or benign verrucas; yet another for the soles of the feet, calluses, warts; the maximum concentration of nitric acid is used for fungus-infected nails.

As the embodiments of the invention are merely descriptive and not limiting, the scope of protection of the present invention will only be limited by the appended claims.

1. A pharmaceutical composition or topical liquid, which destroys pathogenic tissue in order to treat bacterial, viral, carcinogenic and fungal conditions, comprising a mixture of components, characterised in that said aqueous solution is made up by mixing, under acceptable septic conditions at ambient temperature, nitric acid (40% to 90%), mucin (5% to 20%) and ptyalin (5% to 20%) in solution with distilled water as a pharmaceutical vehicle which produces the selective specific coagulation of tissue affected by bacterial, carcinogenic, viral and fungal infections in the dermal tissue, without causing severe side effects in the skin or human mucous membranes where it is applied.

2. A pharmaceutical composition or topical liquid which destroys pathogenic tissue according to claim 1, characterised in that the mixture comprises mucin (5 to 20%) up to a 50% solution in the pharmaceutical vehicle and ptyalin (5 to 20%) up to a 50% solution in the vehicle, plus of nitric acid (40 to 80%) in a 5% to 20% solution in the pharmaceutical vehicle.

3. A pharmaceutical composition or topical liquid which destroys pathogenic tissue according to claim 1, characterised in that the nitric acid is diluted in a proportion of 20% and forms 80% of the total volume of the mixture, 10% of mucin solution and 10% of ptyalin solution.

4. A pharmaceutical composition or topical liquid which destroys pathogenic tissue according to claim 1, characterised in that the nitric acid is diluted in a proportion of 15% and forms 80% of the total volume of the mixture, 10% of mucin solution and 10% of ptyalin solution.

5. A pharmaceutical composition or topical liquid which destroys pathogenic tissue according to claim 1, characterised in that the nitric acid is diluted in a proportion of 5% and forms 80% of the total volume of the mixture, 10% of mucin solution and 10% of ptyalin solution.

6. A pharmaceutical composition or topical liquid which destroys pathogenic tissue according to claim 1, characterised in that the nitric acid is diluted in a proportion of 20% and forms 60% of the total volume of the mixture, 20% of mucin solution and 20% of ptyalin solution.

7. A pharmaceutical composition or topical liquid which destroys pathogenic tissue according to claim 1, characterised in that the nitric acid is diluted in a proportion of 15% and forms 60% of the total volume of the mixture, 20% of mucin solution and 20% of ptyalin solution.

8. A pharmaceutical composition or topical liquid which destroys pathogenic tissue according to claim 1, characterised in that the nitric acid is diluted in a proportion of 10% and forms 60% of the total volume of the mixture, 20% of mucin solution and 20% of ptyalin solution.

9. A pharmaceutical composition or topical liquid which destroys pathogenic tissue according to claim 1, characterised in that the nitric acid is diluted in a proportion of 5% and forms 60% of the total volume of the mixture, 20% of mucin solution and 20% of ptyalin solution.

10. A pharmaceutical composition or topical liquid which destroys pathogenic tissue according to claim 1, characterised in that the nitric acid is diluted in a proportion of 20% and forms 70% of the total volume of the mixture, 15% of mucin solution and 15% of ptyalin solution.

11. A pharmaceutical composition or topical liquid which destroys pathogenic tissue according to claim 1, characterised in that the nitric acid is diluted in a proportion of 15% and forms 70% of the total volume of the mixture, 15% of mucin solution and 15% of ptyalin solution.

12. A pharmaceutical composition or topical liquid which destroys pathogenic tissue according to claim 1, characterised in that the nitric acid is diluted in a proportion of 10% and forms 70% of the total volume of the mixture, 15% of mucin solution and 15% of ptyalin solution.

13. A pharmaceutical composition or topical liquid which destroys pathogenic tissue according to claim 1, characterised in that the nitric acid is diluted in a proportion of 5% and forms 70% of the total volume of the mixture, 15% of mucin solution and 15% of ptyalin solution.

14. A pharmaceutical composition or topical liquid which destroys pathogenic tissue according to claim 1, characterised in that the nitric acid is diluted in a proportion of 20% and forms 90% of the total volume of the mixture, 5% of mucin solution and 5% of ptyalin solution.

15. A pharmaceutical composition or topical liquid which destroys pathogenic tissue according to claim 1, characterised in that the nitric acid is diluted in a proportion of 15% and forms 90% of the total volume of the mixture, 5% of mucin solution and 5% of ptyalin solution.

16. A pharmaceutical composition or topical liquid which destroys pathogenic tissue according to claim 1, characterised in that the nitric acid is diluted in a proportion of 10% and forms 90% of the total volume of the mixture, 5% of mucin solution and 5% of ptyalin solution.

17. A pharmaceutical composition or topical liquid which destroys pathogenic tissue according to claim 1, characterised in that it effects specific coagulation, which is coagulation in the tumoral vasculature associated with the disease, without severe side effects.

18. A pharmaceutical composition or topical liquid which destroys pathogenic tissue, characterised in that it is fixed by the induction of an enzyme at the dermal cell surface of a component of the tumour vasculature, contains a penetration factor and is able to induce selective specific coagulation inside and outside the tumour, the coagulation factor depending on the concentration of the nitric acid, mucin and ptyalin.

19. A pharmaceutical composition or topical liquid which destroys pathogenic tissue according to claim 18, characterised in that the enzymes are taken from the group comprising mucins and ptyalins.

20. A pharmaceutical composition or topical liquid which destroys pathogenic tissue according to claim 18, characterised in that the penetration factor is nitric acid.

21. A pharmaceutical composition or topical liquid which destroys pathogenic tissue according to claim 18, characterised in that it fixes and coagulates the stroma components of the tumour.

22. A pharmaceutical composition or topical liquid which destroys pathogenic tissue according to claim 18, characterised in that it fixes to a base membrane component of the diseased dermal tissue.

23. A pharmaceutical composition or topical liquid which destroys pathogenic tissue according to claim 18, characterised in that it fixes to a platelet activated by the infection.

24. A pharmaceutical composition or topical liquid which destroys pathogenic tissue according to claim 18, characterised in that it fixes to a diseased cell or to a component of the sheath of the cell associated with the infection.

25. A pharmaceutical composition or topical liquid which destroys pathogenic tissue according to claim 18, characterised in that it fixes to a surface receptor of the tumour.

26. A pharmaceutical composition or topical liquid which destroys pathogenic tissue according to claim 18, characterised in that it fixes to the endothelial cell surface of the tumour vasculature and binds operatively with a coagulation factor.

27. Pharmaceutical composition or topical liquid which destroys pathogenic tissue, characterised in that a medication is produced for treating infections of the eyelids, cervix, penis and the rim of the anus.

28. Pharmaceutical composition or topical liquid which destroys pathogenic tissue, characterised in that a drug is produced for treating throat, gum and tongue infections.

29. Pharmaceutical composition or topical liquid which destroys pathogenic tissue, characterised in that a drug is produced for treating foot and scalp infections and benign verrucas.

30. Pharmaceutical composition or topical liquid which destroys pathogenic tissue, characterised in that a drug is produced for treating infections of the sole of the foot, calluses and warts.

31. Pharmaceutical composition or topical liquid which destroys pathogenic tissue, characterised in that a drug is produced for treating fungal infections in the nails.
